(19) United States
(12) Patent Application Publication
(10) Pub. No.: US 2019/0038780 A1
(43) Pub. Date: Feb. 7, 2019

(54) VECTORS AND SYSTEM FOR MODULATING GENE EXPRESSION

(71) Applicant: REGENTS OF THE UNIVERSITY OF MINNESOTA, Minneapolis, MN (US)

(72) Inventors: David A. Largaespada, Moundsview, MN (US); Branden S. Moriarity, Shoreview, MN (US)

(21) Appl. No.: 16/075,218

(22) PCT Filed: Feb. 3, 2017
(86) PCT No.: PCT/US2017/016357

§ 371 Date: Aug. 3, 2018

Related U.S. Application Data
(60) Provisional application No. 62/291,908, filed on Feb. 5, 2016.

(51) Int. Cl. A61K 49/00; C12N 15/113; C12N 15/90; C12N 9/22

(52) U.S. Cl. CPC A61K 49/0008; C12N 15/113; C12N 2320/12; C12N 9/22; C12N 2310/20; C12N 15/90

(57) ABSTRACT

A polynucleotide that modulates transcription from a plurality of genomic targets can include, generally, a polynucleotide encoding a gRNA array and a polynucleotide sequence encoding a nuclease-deficient Cas9 polypeptide. The polynucleotide encoding a gRNA array generally includes polynucleotides encoding at least two gRNAs operably linked to an inducible regulatory sequence.

### CROSS-REFERENCE TO RELATED APPLICATION

This application claims priority to U.S. Provisional Patent Application No. 62/291,908, filed Feb. 5, 2016, which is incorporated herein by reference.

### GOVERNMENT FUNDING

This invention was made with government support under RO3CA201502 awarded by the National Institutes for Health. The government has certain rights in the invention.

### SEQUENCE LISTING

This application contains a Sequence Listing electronically submitted via EFS-Web to the United States Patent and Trademark Office as an ASCII text file entitled “2017-02-01-SequenceListing_ST25.txt” having a size of 8 kilobytes and created on Feb. 1, 2017. The information contained in the Sequence Listing is incorporated by reference herein.

### SUMMARY

This disclosure describes, in one aspect, a polynucleotide for modulating transcription from a plurality of genomic targets. Generally, the polynucleotide includes a polynucleotide encoding a gRNA array and a polynucleotide sequence encoding a nuclease-deficient Cas9 polypeptide. The polynucleotide encoding a gRNA array generally includes polynucleotides encoding at least two gRNAs operably linked to an inducible regulatory sequence.

In some embodiments, an enzyme-cleavable linker sequence links the polynucleotide encoding the first gRNA and the polynucleotide encoding the second gRNA.

In some embodiments, the nuclease-deficient Cas9 polypeptide comprises a fusion polypeptide including a transcription activating domain. In some of these embodiments, the transcription activating domain can include VP64.

In some embodiments, the nuclease-deficient Cas9 polypeptide can include a transcription repressing domain. In some of these embodiments, the transcription repressing domain can include a Krüppel associated box domain.

In some embodiments, the gRNA array can include at least 5 gRNAs.

In another aspect, this disclosure describes a method of modulating expression of a plurality of genomic target coding regions in a cell. Generally, the method includes introducing into the cell any embodiment of the polynucleotide summarized above, wherein gRNAs in the array target the genomic target coding regions, and inducing transcription of the gRNA array.

In some embodiments, the method involves modulating expression of two or more genomic target coding regions simultaneously.

In some embodiments, the method can further include screening the modulated expression of the genomic target coding regions for a change in phenotype.

In some embodiments, the method can further include identifying mRNA targets of a particular phenotype.

In some embodiments, the method can further include identifying causal cancer genes.

In some embodiments, the method can further include overexpressing a genomic target coding region that encodes a polypeptide of interest. In some of these embodiments, the method further includes isolating at least a portion of the polypeptide of interest.

In some embodiments, the method can further include altering biochemical pathways to favor biosynthesis of a compound of interest. In some of these embodiments, the method can further include isolating at least a portion of the compound of interest.

In some embodiments, the method can further include generating a synthetic CRISPR immune system to increase resistance of the cell to infection by a virus.

In some embodiments, the method can further include activating a cellular pathway in a therapeutic cell to increase the therapeutic cell's therapeutic activity.

In another aspect, this disclosure describes a method for generating a genetically modified organism. Generally the method includes introducing into cells of the organism any embodiment of the polynucleotide summarized above, wherein gRNAs in the array target the genomic target coding regions, and inducing transcription of the gRNA array.

In some embodiments, the organism can be a mouse.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Diagram of the CRISPR locus in Pseudomonas aeruginosa UCBPP-PA14. The CRISPR locus is flanked by arrays of spacers linked by target sequences (SEQ ID NO:2) of the sequence specific RNA nuclease Csy4.

FIG. 2. Plasmids required for Golden Gate assembly of up to 10 gRNAs linked by Csy4 sites (Left). Example Golden Gate assembly of 4 gRNAs (right).

FIG. 3. CELI results of 10 gRNA array targeting 10 genes. (A) Diagram of gRNA array. (B) Cells treated with Cas9 and 10 gRNA array. (C) Cells treated with Cas9, Csy4, and the 10 gRNA array.

FIG. 4. (A) Diagram of gRNA arrays in pENTR1 and pENTR2. (B) Diagram of PB-Dual-DEST-Sp dCas9 Activator vector. (C) Final PB-Dual-gRNA-Array-Sp dCas9 Activator vector. Sp: S. pyogenes DEST: Gateway destination cassette, TRE: tertracycline responsive element, rtTA: reverse tetracycline transactivator, Puro: puromycin, ITR: piggyBac inverted terminal repeates, 2A: ribosomal skip sequence P2A.

FIG. 5. (A) Schematic diagram of the schema to identify highly functioning gRNAs. (B) Hypothetical single cell RNA sequencing results from a cell expressing 5 gRNAs targeting genes A-E and the dCAS9:VP64 fusion. *** Indicates gRNAs that robustly induce expression of their target gene.

FIG. 6. Diagram of the base pGG (left) and pENTR-ACPT (right) plasmids highlighting the type IIS restriction enzymes used for protospacer oligonucleotide ligation (BsaI) and golden gate assembly (BsmBI). In addition, the pGG cassette contains a filler sequence that is removed upon enzyme digestion and a 5′ Csy4 site for array processing once assembled and expressed. A terminal Csy4 site was included in the pENTR-ACPT cassette to remove additional plasmid sequence from the terminal gRNA when expressed and a LacZ gene that is removed upon golden gate assembly to allow for blue/white colony selection.

FIG. 7. Gene editing frequency of pol III driven 10 gRNA array. (A) Diagram depicting the plasmid vectors transfected into HEK293T cells to induce targeted DSBs using a pol III driven 10 gRNA array combined with Cas9 and Csy4. (B) Results of CRISPR/Cas9 editing at each of the 10 gRNA target sites when using gRNA arrays with a 20 bp or 28 bp Csy4 target sequence. (C) Results of surveyor nuclease assay performed on genomic DNA of HEK293T cells transfected with a 10 gRNA array and Cas9 with or without Csy4 three days post transfection. Mutation frequencies were assessed by Surveyor Nuclease assay with means of triplicate measurements shown. P2A: ribosomal skip sequence; BGH pA: bovine growth hormone polyadenylation signal; CAG: strong mammalian promoter comprised of cytomegalovirus (CMV) early enhancer element, the first exon and intron of chicken beta-actin gene, and the splice acceptor of the rabbit beta-globin gene.

FIG. 8. Comparison of gene editing frequency of pol II-driven and pol III-driven three-gRNA array, five-gRNA array, and seven-gRNA array. Line graphs (left) depicting the gene editing frequency of each gRNA when expressed as individual gRNAs transcribed from the standard U6 pol III promoter (dots) or in a single three-gRNA array (top), five-gRNA array (middle), or seven-gRNA array (bottom) transcribed from the standard U6 pol III promoter, CMV promoter with BGH polyadenylation signal, and CAG promoter with BGH polyadenylation signal three days post transfection. Bar graphs (right) depicting the average gene editing frequency of the three-gRNA array (top), five-gRNA array (middle), seven-gRNA array (bottom) expressed from each promoter normalized to the editing frequency of each individual gRNA transcribed from the standard U6 pol III promoter. Mutation frequencies were assessed by Surveyor Nuclease assay with means of triplicate measurements shown.

FIG. 9. Modified golden gate assembly plasmid library. Diagram depicting the cloning strategy to remove the U6 promoter from the pENTR-ACPT 1-10 plasmids used for gRNA array assembly. Plasmids were treated with DraI (blunt) and subsequently self-ligated and sequence verified.

FIG. 10. Comparison of gene editing frequency of pol II and pol III driven gRNA arrays. (A) Diagram depicting the plasmid vectors transfected into HEK293T cells containing pol II or pol III promoters driving transcription of a 10 gRNA array. (B, left panel) Graph depicting the gene editing frequency of each gRNA when expressed as individual gRNAs transcribed from the standard U6 pol III promoter or in a single 10 gRNA array transcribed from the standard U6 pol III promoter, CMV promoter with BGH polyadenylation signal, and CAG promoter with BGH polyadenylation signal assessed three days post transfection. (B, right panel) Bar graph depicting the average gene editing frequency of the 10 gRNA array expressed from each promoter normalized to the editing frequency of each individual gRNA transcribed from the standard U6 pol III promoter. Mutation frequencies were assessed by Surveyor Nuclease assay with means of triplicate measurements shown. P2A: ribosomal skip sequence; BGH pA: bovine growth hormone polyadenylation signal; CMV: cytomegalovirus; CAG: strong mammalian promoter comprised of CMV early enhancer element, the first exon and the first intron of chicken beta-actin gene, and the splice acceptor of the rabbit beta-globin gene. **P<0.001, ***P<0.0001, Student's t test. Error bars, s.d.

FIG. 11. Enhanced multiplex editing using gRNA arrays. (A) Diagram depicting the plasmid vectors transfected into HEK293T cells to compare gene editing by multiplexing 10 standard U6-gRNA plasmids and a 10 gRNA array. (B) Bar graph depicting the gene editing frequency at each of 10 gRNA target sites three days post transfection using multiplexed individual U6-gRNA plasmids or 10 gRNA array encoding the same gRNAs. Mutation frequencies were assessed by Surveyor Nuclease assay with means of triplicate measurements shown. P2A: ribosomal skip sequence; BGH pA: bovine growth hormone polyadenylation signal; CAG: strong mammalian promoter comprised of CMV early enhancer element, the first exon and the first intron of chicken beta-actin gene, and the splice acceptor of the rabbit beta-globin gene. **P<0.001, ***P<0.0001, Student's t test. Error bars, s.d.

FIG. 12. Stable expression of the CRISPR/Cas9 based gRNA array system. (A) Diagram depicting gateway ready DNA transposon vector for expression of all components of the gRNA system for multiplex editing. (B) Results of gene editing at all 10 gRNA target sites seven days post transfection and puromycin selection in HEK293T cells. Mutation frequencies were assessed by Surveyor Nuclease assay with means of triplicate measurements shown.

FIG. 13. Golden Gate assembly of MS2 gRNA arrays. (A) Diagram of the base pGG-MS2 (left) and pENTR-ACPT (right) plasmids highlighting the type IIS restriction enzymes used for protospacer oligonucleotide ligation (BsaI) and Golden Gate assembly (BsmBI). In addition, the pGG-MS2 cassette contains a filler sequence that is removed upon oligonucleotide ligation and a 5′ Csy4 site for array processing once assembled and expressed. A terminal Csy4 site was included in the pENTR-ACPT cassette to remove additional plasmid sequence from the terminal gRNA when expressed and a LacZ gene that is removed upon Golden Gate assembly to allow for blue/white colony selection. (B) Diagram of the final 10 pGG-MS2 and 10 pENTR-ACPT plasmids for assembly of arrays containing 1-10 gRNAs. The gateway attL1/2 sites of pENTR-ACPT plasmids have been left out for simplicity.

FIG. 14. Multiplexed gene activation using the SAM system with gRNA arrays. (A) Diagram depicting the elements encoded in plasmids used for multiplex gene activation using the SAM system combined with gRNA arrays containing MS2 sequences. (B) RT-PCR results of gene activation at five gRNA target sites three days post transfection using individual U6-gRNAs or a gRNA array containing all five gRNAs (left). Average gene activation using either approach is also shown, demonstrating no difference in gene activation using single U6-gRNA plasmids or gRNA arrays (right).

### DETAILED DESCRIPTION OF ILLUSTRATIVE EMBODIMENTS

This disclosure describes a system to induce, repress, knockout, or otherwise modify numerous genes at one time in a single cell, including in vivo. There have been numerous reports and advances in the use of nuclease deficient Cas9 (dCas9) for targeted activation or repression of gene expression in human cells. For example, dCas9 fused to the transcriptional activation VP64 domain can induce targeted gene activation, which can be enhanced when combined with p65/HSF1 recruitment, providing optimal and robust gene activation. Alternatively, dCas9 fused to a specific p300 domain can robustly activate gene expression through epigenetic modifications. dCas9 targeted to a promoter region can interfere with transcription on its own and this gene repression is enhanced when a Krüppel associated box (KRAB) domain is fused to dCas9.

The system described herein allows one to stably or transiently deliver numerous gRNAs targeting coding regions, miRNAs, and/or lncRNAs to a single cell at one time, along with the appropriate dCas9 fusion. To further control this system, in the event the activation or repression is toxic or lethal to cells, the dCas9 fusion can be under the control of an inducible operon such as, for example, the tetracycline operon.

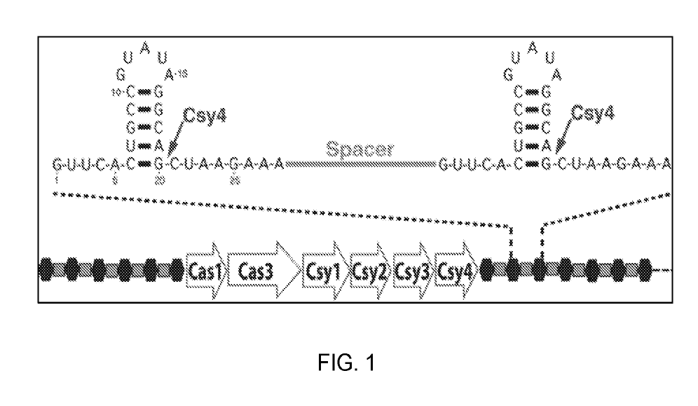

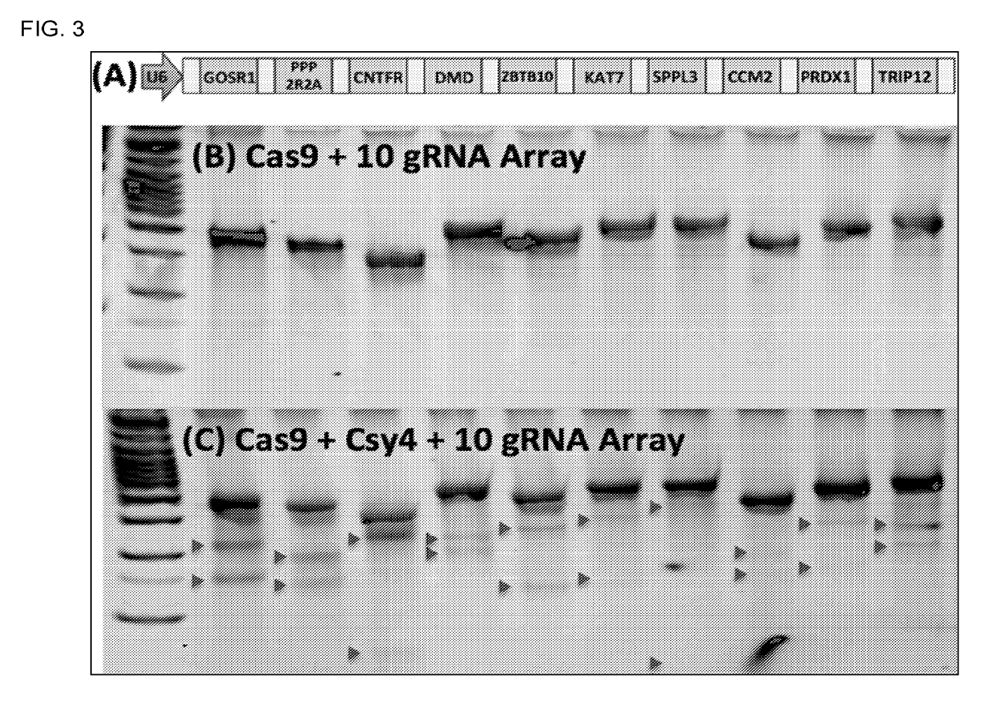

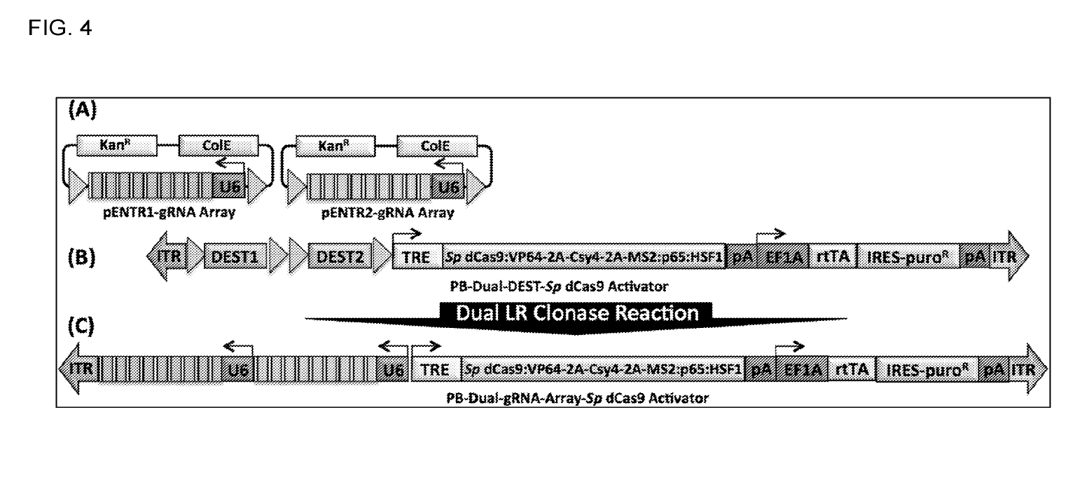

In order to accommodate the stable delivery of many gRNAs at once, the system involves linking many gRNAs in a gRNA array expressed from a single U6 promoter, analogous to what is observed in nature (FIG. 1). The gRNAs are assembled using Golden Gate cloning with a library of validated plasmids or PCR products or annealed DNA oligonucleotides (FIG. 3). The assembled arrays contain sites in between each gRNA that are identified and cleaved by the site-specific RNA nuclease Csy4, which can be co-expressed with the dCas9 fusions. We have been able to assemble arrays ranging from 2-10 gRNAs with >99% efficiency and all assembled vectors sequence perfectly via standard Sanger sequencing. To validate the functionality of our assembled arrays, we induced double strand breaks at 10 independent genomic sites by transfecting HEK293T cells with a plasmid encoding Cas9 nuclease, Csy4, and a 10 gRNA array. CELI assay performed on genomic DNA from treated cells demonstrated detectable cutting at all 10 target genes only when both Cas9 and Csy4 were co-delivered (FIG. 4).

It is also possible to further clone two (or more) arrays of 10 together to have 20 (or more) gRNAs expressed form a single promoter. Thus, in certain embodiments, the upper limit of the number of gRNAs while maintaining robust Csy4 processing and Cas9 targeting can be, for example, no more than 200 gRNAs such as, for example, no more than 150 gRNAs, no more than 125 gRNAs, no more than 100 gRNAs, no more than 75 gRNAs, no more than 50 gRNAs, no more than 45 gRNAs, no more than 40 gRNAs, no more than 35 gRNAs, no more than 30 gRNAs, no more than 25 gRNAs, no more than 24 gRNAs, no more than 23 gRNAs, no more than 22 gRNAs, no more than 21 gRNAs, no more than 20 gRNAs, no more than 19 gRNAs, no more than 18 gRNAs, no more than 17 gRNAs, no more than 16 gRNAs, no more than 15 gRNAs, no more than 14 gRNAs, no more than 13 gRNAs, no more than 12 gRNAs, no more than 11 gRNAs, no more than 10 gRNAs, no more than nine gRNAs, no more than eight gRNAs, no more than seven gRNAs, no more than six gRNAs, or no more than five gRNAs. Thus, in some cases, the system can involve delivery of, for example, up to 20 gRNAs. In certain embodiments, the system can involve delivery of 5-10 gRNAs.

For stable delivery of the doxycycline inducible system, one can use, for example, any suitable transposon vector system or viral vector system. In some cases, one can use a piggyBac transposon vector system, which is a cut and paste DNA transposon capable of integrating cargo of greater than 100 kb in size. The piggyBac transposon system can provide one or more benefits over standard lentiviral vectors. For example, lentiviral vectors can be incapable of faithfully delivering cargo containing many repeat regions, they can have a limited cargo capacity (e.g., up to approximately 12 kb), and they can be more time consuming to generate compared to plasmids. The vector can be designed to include dual LR Clonase Gateway (Life Technologies Corp., Carlsbad, Calif.) ready sites for simple and efficient cloning of gRNA arrays (FIG. 4).

When using a CRISPR activation and repression system, the placement of gRNAs relative to the promoter region of the gene influences the extent of gene modulation. One can design, for example, 5-10 gRNAs for a given target and identify the gRNA or gRNAs that activate or repress transcription of the target to the desired level. One can target multiple coding regions (e.g., 10-20) by, for example, integrating a gRNA for each target coding region.

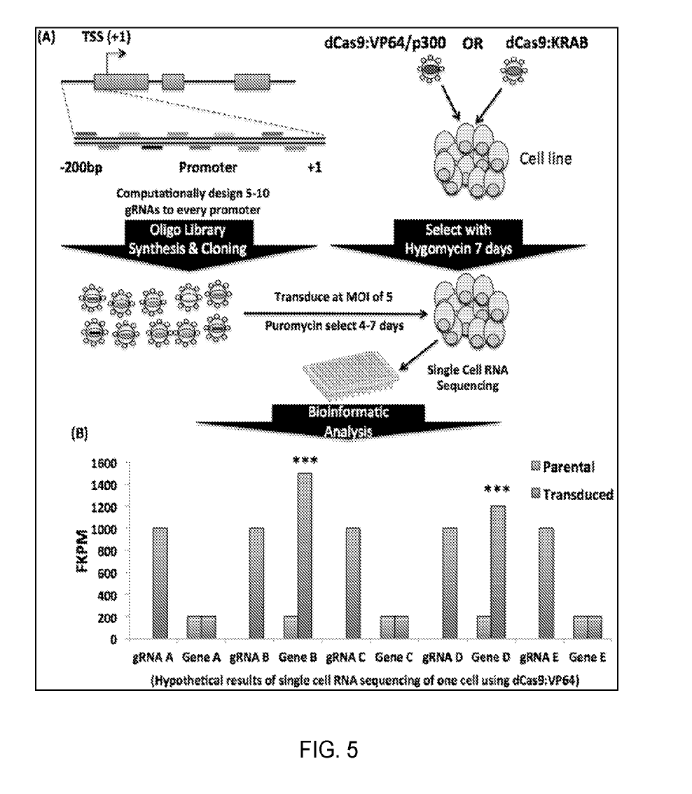

This disclosure describes a schema to identify, in a high throughput manner, gRNAs that modulate gene expression to a desired degree. The schema involves using single cell RNA sequencing. In an exemplary embodiment, 5-10 gRNAs are computationally designed to the promoter region of each gene, lncRNA, or miRNA of interest (FIG. 5A). The gRNAs can be synthesized into oligonucleotides and cloned into lentiviral vectors as previously described (Nissim et al., 2014, Mol. Cell 54:698-710). Stable cell lines expressing an appropriate vector (e.g., dCas9:VP64/p300 or dCas9:KRAB) can be transduced with the gRNA library and selected and expanded. The cells may be transduced at a multiplicity of infection (MOI) such that each cell gets multiple gRNAs. The transduced cells can be transcriptionally profiled using single cell RNA sequencing. Within each cell, one can categorize each gRNA that is expressed and determine if the expressed gRNA correlates with significant changes in transcript expression of its cognate gene (FIG. 5B). This method allows one to identify gRNAs for each target gene, lncRNA, and miRNA, and have a quantitative measure of how well each gene-specific gRNA functions for gene activation or repression.

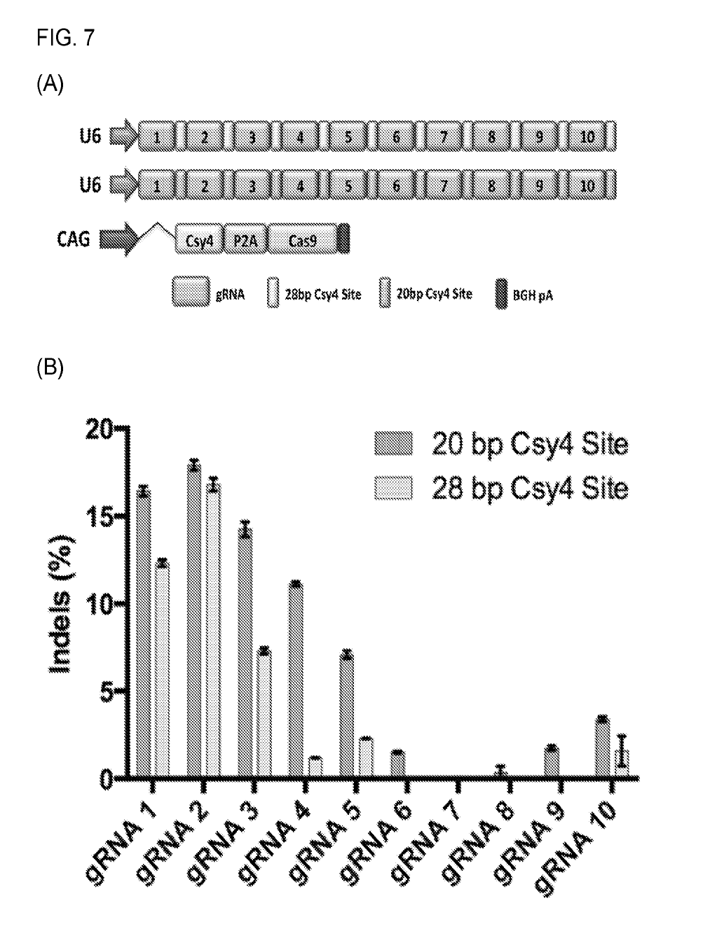

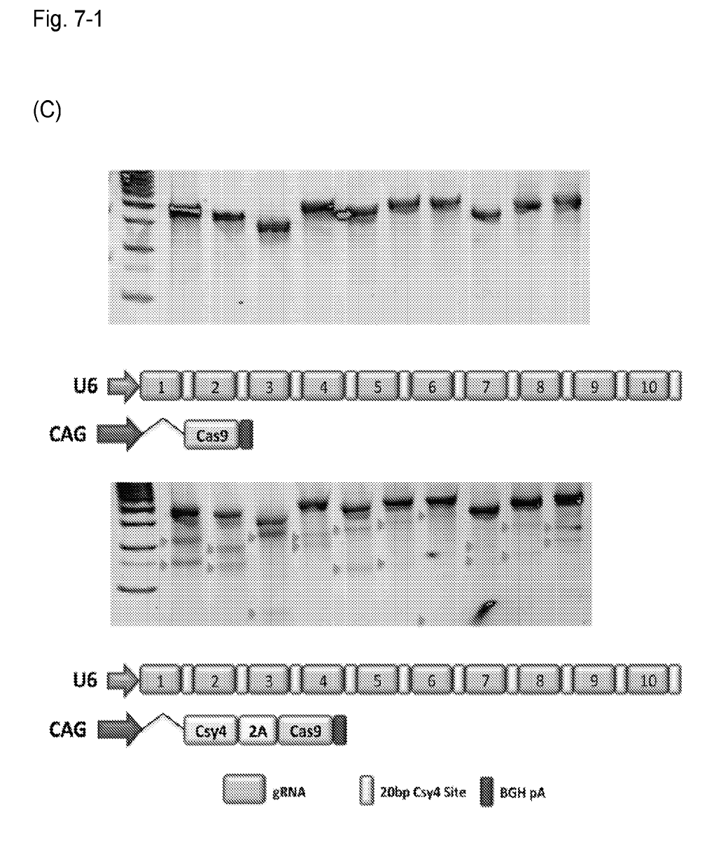

For example, HEK293T cells were transfected with gRNA arrays of three, five, seven or ten gRNAs and a plasmid expressing Cas9 alone or Cas9 linked to the human codon-optimized Csy4 ribonuclease (Tsai et al., 2014. Nat. Biotechnol., 32:569-576) via a P2A element (FIG. 7A). Negligible editing was observed without expression of Csy4 to process the array into individual gRNAs, confirming the necessity of Csy4 for array processing (FIG. 7C). The results of nuclease activity for the 10 gRNA array transfected with Cas9-P2A-Csy4 demonstrated detectable rates of editing with the first four gRNAs in the array and then the editing diminished to nearly undetectable levels at gRNA 7 and gRNA 8, but editing was again observed with the gRNA 9 and gRNA 10 (FIG. 7B).

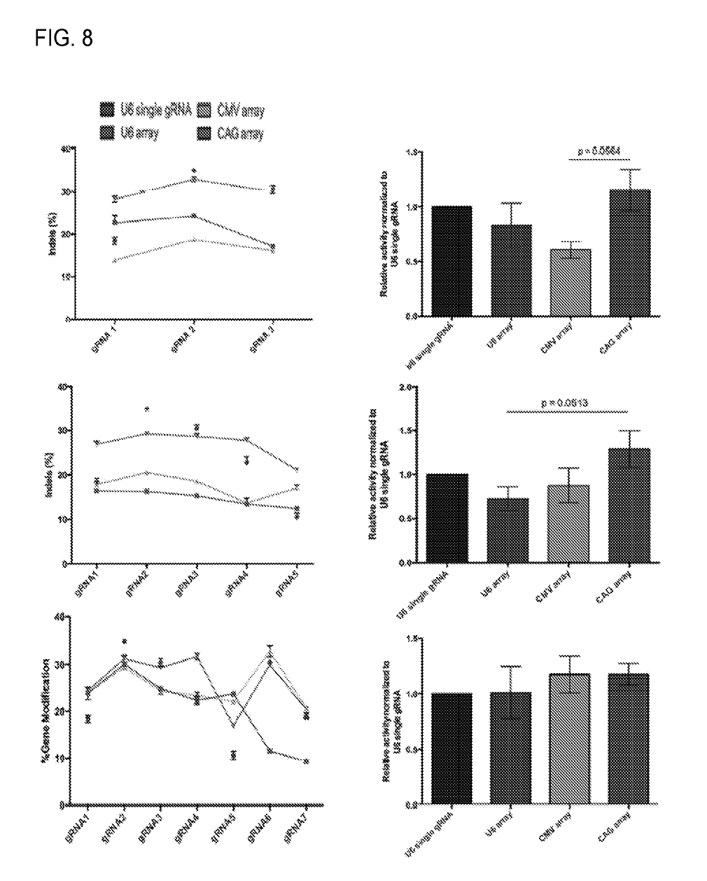

Previous reports have identified Cys4 target sites of 20 bp and 28 bp in length (Tsai et al., 2014. Nat. Biotechnol., 32:569-576; Nissim et al., 2014. Mol. Cell 54:698-710). Thus, an additional set of pGG1-10 and pACPT plasmids were generated harboring the 28 bp Csy4 target site and again assembled the 10 gRNA array expressed via the U6 pol III promoter. The gRNA array containing the 20 bp Csy4 site produced higher levels of gene editing at all 10 target sites, indicating the 20 bp Csy4 site may be more efficiently cleaved by Csy4 than the 28 bp sequence (FIG. 7B). Editing frequencies using U6 driven three-gRNA arrays, five-gRNA arrays, and seven-gRNA arrays also induced detectable gene editing. (FIG. 8).

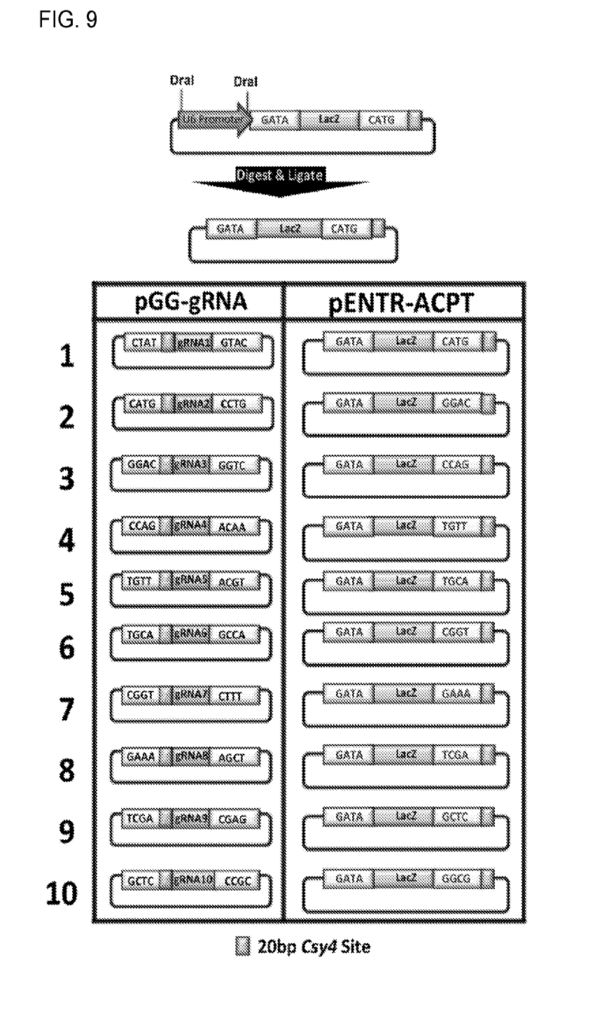

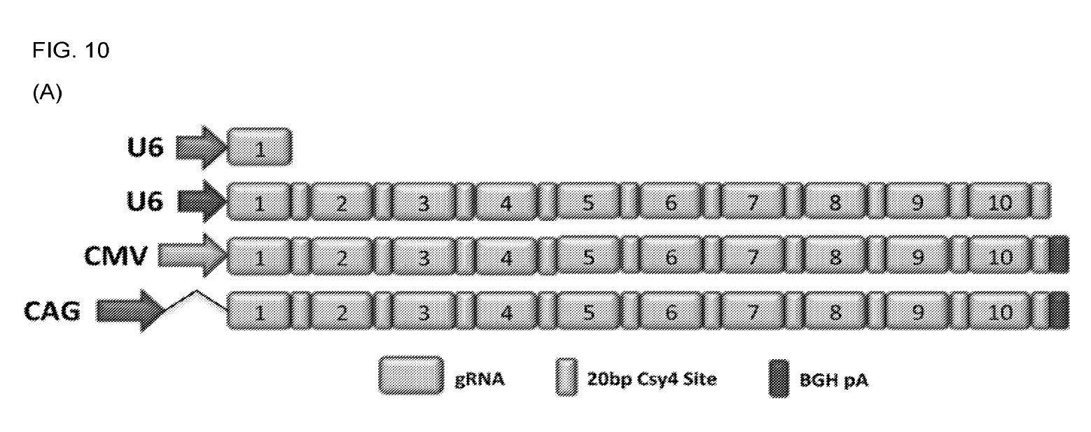

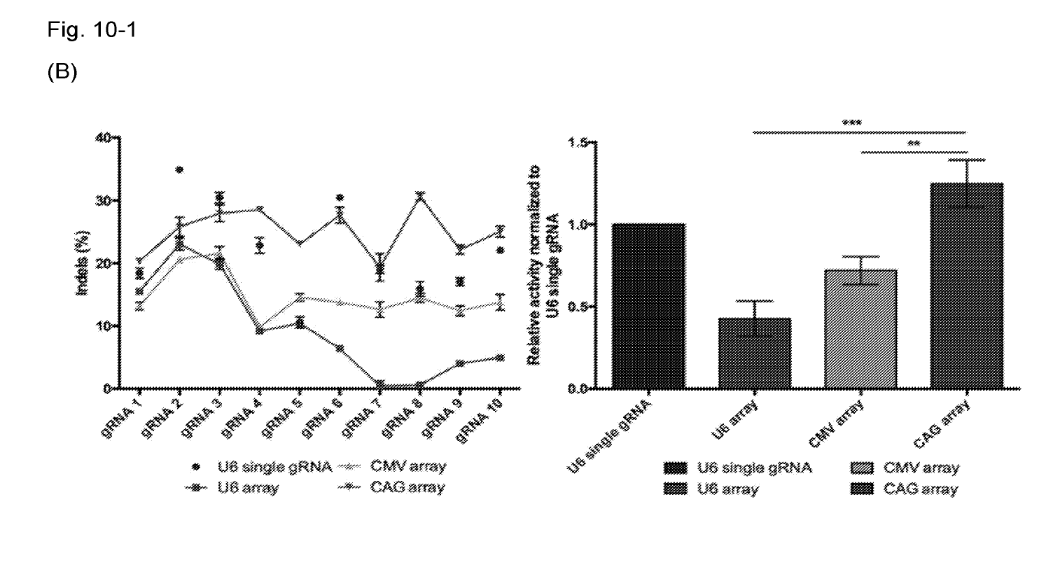

Editing frequencies were increased by removing the U6 promoter from the pACPT 1-10 plasmids, as illustrated in FIG. 9, and again assembled an array of 10 gRNAs that were subsequently cloned into a vector containing the strong pol II CMV promoter with a poly adenylation sequence (FIG. 10A). The array was transfected into HEK293T and demonstrated improved gene editing frequencies overall, with approximately 10%-21% gene editing across all gRNA targets (FIG. 10B, CMV array vs. U6 single gRNA). The 10 gRNA array was expressed from the very strong intron-containing pol II CAG promoter with a poly adenylation sequence (FIG. 10A, CAG). The rates of gene editing using the CAG promoter were significantly higher than the individual U6-gRNA editing (FIG. 10B, CAG array vs. U6 single gRNA, P>0.0002). The effect of the promoter in driving the expression of three-gRNA arrays, five-gRNA arrays, and seven-gRNA arrays were tested and increased gene editing was observed using the CAG promoter (FIG. 8). These results demonstrate gRNA arrays expressed from strong pol II promoters with polyadenylation sequences enhance gene editing frequencies to levels as high or higher than the gene editing levels observed with individual standard U6-gRNA plasmids.

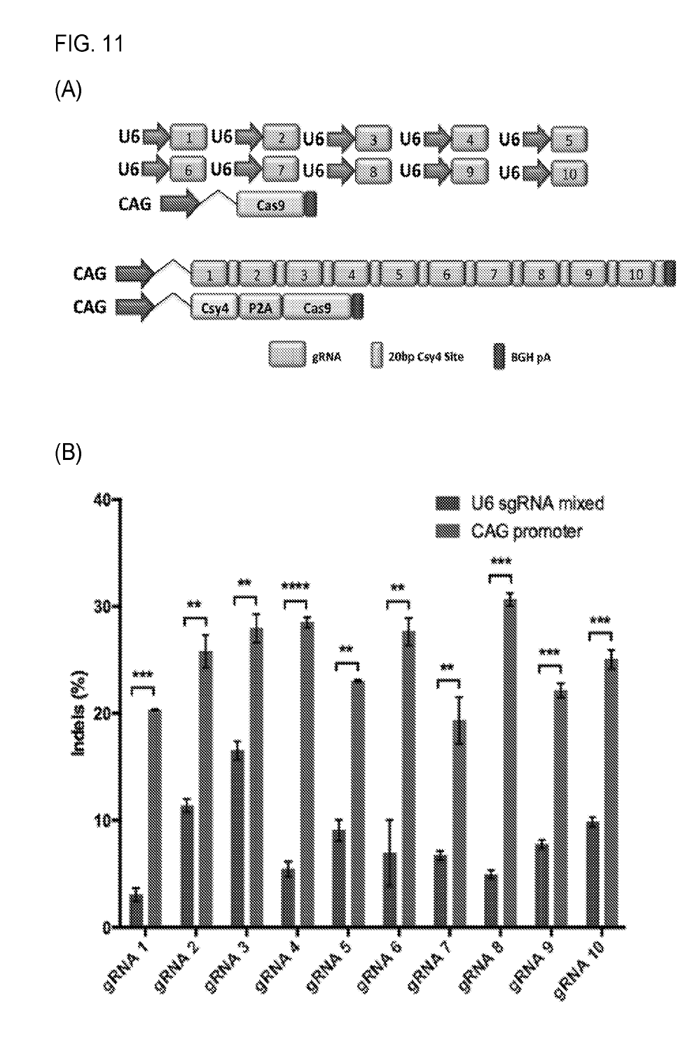

Effects of transfecting a cell with numerous U6-gRNA-containing plasmids include toxicity and low transfection efficiency. Thus, the gRNA array plasmids were tested head-to-head with standard multiplexed U6-gRNA plasmid delivery (FIG. 11A). Cells were transfected with either each of the 10 individual U6-gRNA plasmids along with Cas9 or the gRNA array and Csy4/Cas9 vector. In both cases, the total amount (in micrograms) of transfecting plasmid was the same. This resulted in no obvious toxicity and analysis of gene editing efficiency at all 10 target sites was significantly higher using the gRNA array approach (FIG. 11B). The average editing efficiency for 10 individual U6-gRNA plasmids was significantly lower (8.2%) compared to with the gRNA array (25.0%). These data demonstrate the gRNA array system is a superior approach to the use of numerous multiplexed U6-gRNA plasmids.

Stable Expression of the CRISPR/Cas9 Based gRNA Array System

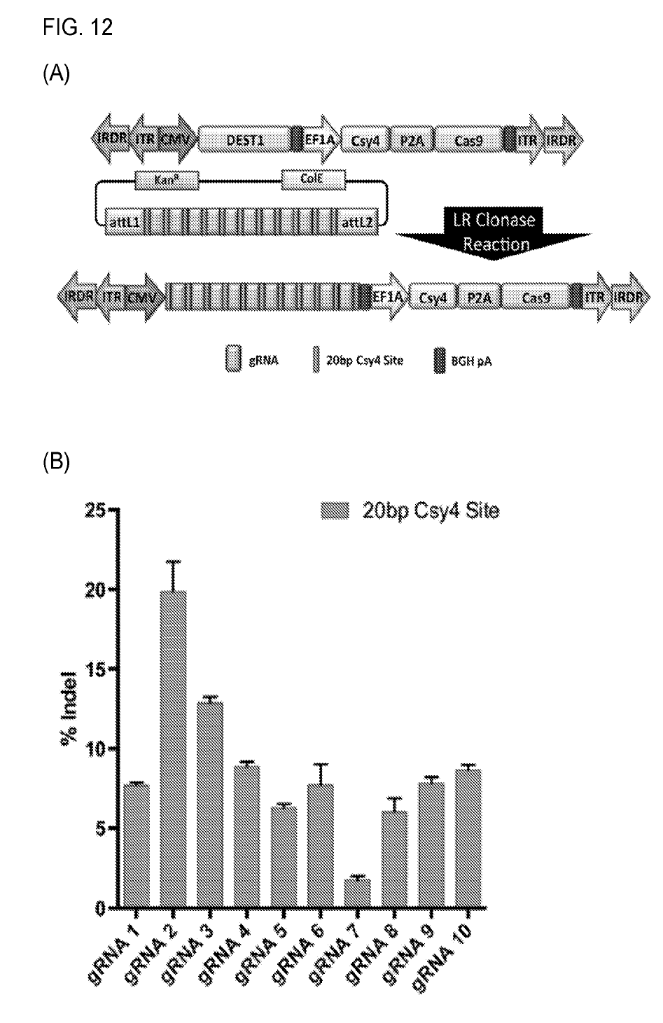

An all-in-one gateway-ready transposon vector, compatible with both piggyBac and Sleeping Beauty systems, was developed to investigate the ability to stably express the gRNA array system in human cells (FIG. 12A). The 10 gRNA array was transferred to the transposon vector and stably integrated the transposon into HEK293T cells using piggyBac transposase. Surveyor nuclease assay demonstrated gene editing at all target sites of the 10 gRNA array after puromycin selection (FIG. 12B). These data demonstrate that DNA transposons can be used to successfully deliver functional gRNA arrays to human cells.

Multiplex SAM Activation Using gRNA Arrays

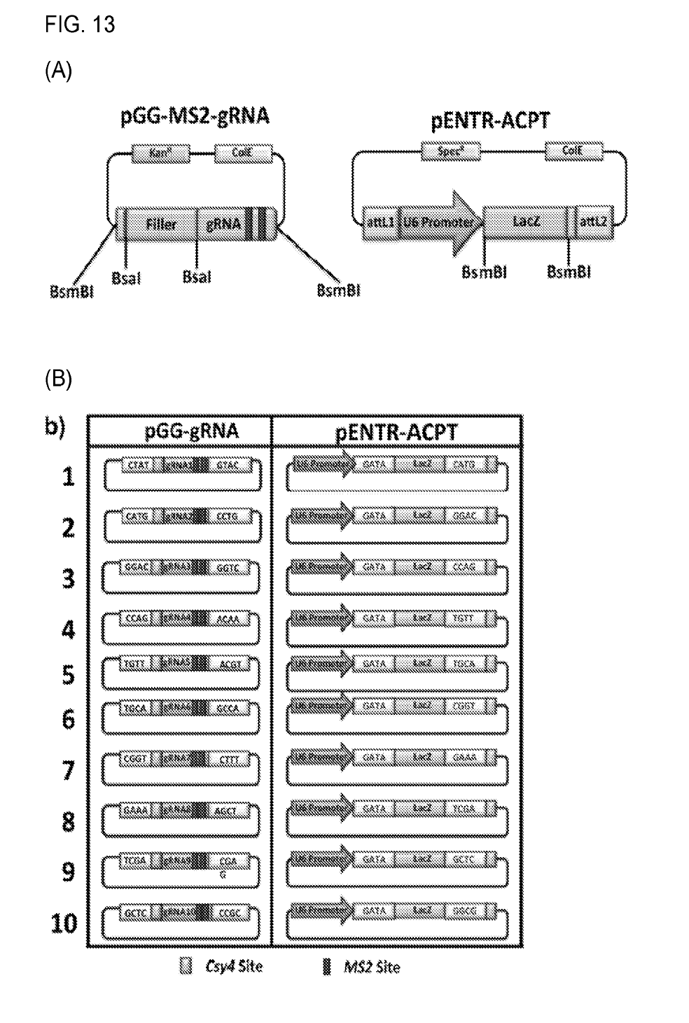

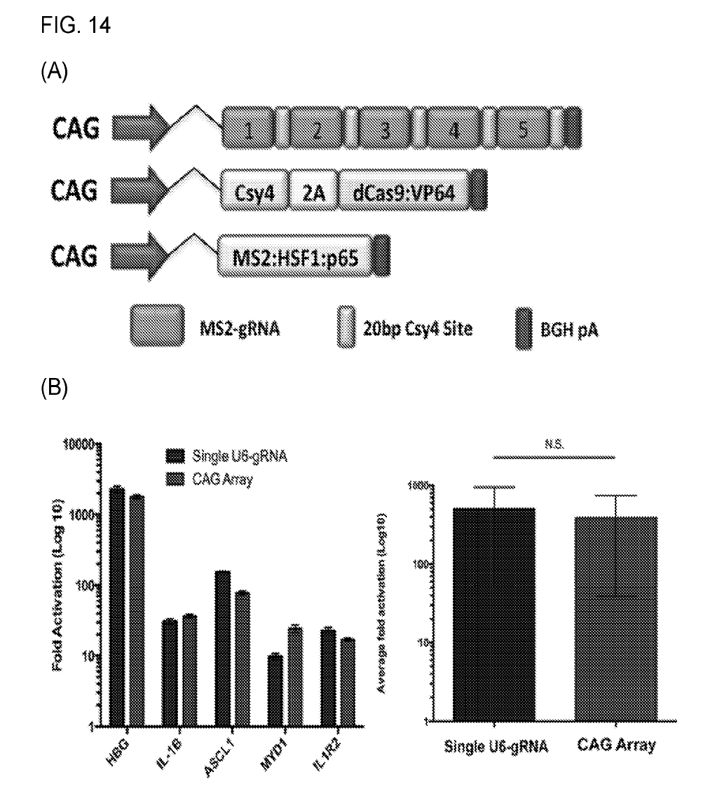

A set of pGG1-10 vectors with gRNAs containing two MS2 binding sites were generated to allow for multiplex gene activation using gRNA arrays (FIG. 13A). These gRNAs are compatible with the SAM activation system (Konermann et al., 2015. Nature 517(7536):583-588). This system was used to generate a gRNA activation array containing five previously validated gRNAs used for gene activation (Konermann et al., 2015. Nature 517(7536):583-588) (FIG. 14A). HEK293T cells were transfected with individual U6-MS2-gRNAs, dCas9-VP64, or MS2:p65:HSF1 plasmids to assess standard gene activation with the SAM system using individual U6 gRNAs (FIG. 14B). These activation results were then compared with gene activation in cells transfected with the MS2 gRNA array, Csy4/dCas9-VP64, or MS2:HSF1:p65 plasmids. Robust levels of gene activation were observed in both systems and the level of activation was not significantly different between using individual U6-MS2-gRNA plasmids or the MS2 gRNA array (FIG. 14B). These results demonstrate that MS2 gRNA arrays are amenable to multiplex gene activation and produce levels of activation on par with individual U6-MS2-gRNA plasmids.

Thus, this disclosure describes assembly of CRISRP/Cas9 gRNA arrays capable of expressing multiple gRNAs from a single promoter. The gRNA arrays are effectively processed by Csy4 ribonuclease and high rates of gene editing can be detected at all gRNA target sites when the gRNA array is expressed from a suitable promoter. In one exemplary embodiment, the array can be expressed from the pol II CAG promoter containing a polyadenylation sequence. Moreover, gene editing frequencies are higher when using pol II-driven gRNA arrays compared to the individual standard U6-gRNA plasmids, especially when multiplexing numerous U6-gRNA plasmids. It is also possible to stably express the gRNA arrays in cultured mammalian cells when delivered using DNA transposons.

One characteristic of using the CAG promoter to drive gRNA array expression was increased gene editing frequencies compared to individual U6-gRNAs plasmids, which is the most commonly used format of the CRISPR/Cas9 system. This is unexpected as the U6 promoter has been shown to be highly efficient at transcription of gRNAs with nearly a log-fold higher expression compared to, for example, CMV. Without wishing to be bound by any particular theory, the CAG promoter may produce larger amounts of transcript compared to the standard U6 promoter. Another possible reason for enhanced editing using the gRNA arrays described herein may be due to the use the Csy4 enzyme. Csy4 may protect the gRNAs from degradation that normally occurs from endogenous non-specific RNases in the cytoplasm, providing a larger window of time for Cas9 to bind the gRNA and induce targeted DSBs. Alternatively, Csy4 may directly interact with Cas9 to enhance gRNA loading after gRNA array processing.

Another strategy for increasing gene editing frequencies involves modifying the gRNAs to include a nuclear localization sequence (e.g., Zhang et al., 2014. Mol Cell Biol 34(12):2318-2329). A nuclear localization sequence can protect the gRNAs from degradation by cytoplasmic nucleases by localizing the gRNAs in the nucleus of the cell.

While described herein in the context of an exemplary embodiment in which the gRNA array platform for spCas9 employs standard and MS2-containing chimeric gRNA backbones, similar platforms employing other CRISPR orthologs (e.g., Neisseria meningitidis Cas9 and Staphylococcus aureus Cas9), other modified gRNA backbones, and/or other CRISPR systems (such as Cpf1). Moreover, it is possible to generate Golden Gate assembly libraries to mix and match various gRNA backbones to use multiple orthologs simultaneously. For instance, Sp dCas9-VP64 can be used for gene activation combined with Sa dCas9-KRAB for gene repression using a gRNA array containing both Sp and Sa specific gRNA backbones. As another example, one can use Cpf1 for enhanced multiplex genome engineering exploiting the character of Cpf1 having both DNase and RNase activity. The DNase activity of Cpf1 can be exploited to induce sequence specific DSBs and its RNase function can be exploited to process the transcribed CRISPR arrays into individual gRNAs. Thus, Cpf1 may be used to deliver analogous functions of Cas9 and Cys4 in a single protein.

Another character of gRNA array technology that may be useful in certain applications is the ability to use in vitro transcribed (IVT) RNA encoding the gRNA array. This approach may be especially desirable for multiplexed editing of primary human lymphocytes, such as T cells. Plasmid DNA is toxic to primary lymphocytes and thus the use of IVT gRNA arrays can allow for multiplex gene editing of primary human cell types for research and therapy.

The methods described herein can be used in connection with any application that involves modulating expression of many genes at one time in a single cell. Thus, the methods may be used in connection with, for example, inducing transdifferentiation, screening candidate genes for a given phenotype, identifying transcription factor targets of a given phenotype, identifying miRNA targets of a given phenotype, identifying causal cancer genes in amplified or deleted regions in cancer, super overexpression of a gene or cDNA for the purpose of protein production, altering biochemical pathways to favor the production of a given compound, modeling cancer development/metastasis/drug resistance, generating synthetic CRISPR immune systems to protect cells (such as immune cells or any cell) from invading viruses, activating cellular pathways in therapeutic cells to improve therapeutic effects (such as cells for the purpose of gene therapy or immunotherapy).

The ability to deliver multiple gRNAs at one time allows the CRISPR/Cas9 system to easily perform multiplex genome editing. By implementing multiplexed CRISPR gRNAs, one can target, for example, up to 50 coding regions for deletion and/or activation. The CRISPR system is highly effective at inducing large deletions (e.g., 300 kb) and can achieve more massive deletions (e.g., >30 Mb, unpublished result), opening the possibility to use this technology for large-scale chromosome engineering. This can allow for functional genomics studies targeting commonly deleted chromosomal regions of human cancer in the mouse. The analogous regions commonly lost in human cancers can be targeted for deletion in segments in the mouse using the MCC system. Deletion of specific regions harboring critical genes may lead to tumor development or progression, thereby functionally identifying the critical genes in large regions that drive tumor formation. In addition to large chromosomal deletions, targeted nucleases have been used to generate common translocations observed in human cancer leading to the production of oncogenic fusion proteins, such as EWSR1-FLI1 and NPM1-ALK fusions found commonly in Ewing sarcoma and anaplastic large cell lymphoma (ALCL), respectively. These fusions could be generated de novo in mouse somatic cells using the Merkel Cell Carcinoma (MCC) model, which may be more accurate than simply over expressing the human fusion cDNA as is typically done to study these oncogenic fusions. These are just a few experimental possibilities with the MCC model, demonstrating the potential power and utility of the system to the field of cancer research and other diseases.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### Examples

### CRISPR Expression Vector Construction

Inducible CRISPR vectors were designed using published sequences for all elements, such as Cas9, Csy4, rtTA, Puro, EF1A, etc. Designed sequences were then ordered as gBLOCKs (IDT) in 2 kb fragments and assembled using Gibson assembly (New England Biolabs, Inc., Ipswich, Mass.), following the manufacturer's instructions.

### Golden Gate Platform Assembly

Optimal overhangs for the assembly of up to 10 unique gRNAs were determined bioinformatically as previously described (Cermak et al., 2011, Nucleic Acids Res 39(12):e82). Csy4 site sequences have been previously described (Nissim et al., 2014, Mol Cell 54:698-710; Tsai et al., 2014, Nature Biotechnol 32:569-576). Both the 20 bp (5′-guucacugccguauaggcag-3′; SEQ ID NO:1) and the 28 bp (5′-guucacugccguauaggcagcuaagaaa-3′; SEQ ID NO:2) handle region containing Csy4 sites were used to determine optimal sequences. These 10 gRNA fragments with appropriate BsaI type IIS restriction enzyme sites for Golden Gate cloning were ordered as gBLOCK fragments from IDT and included attB sequences on both ends for subsequent BP Clonase reaction using Gateway cloning, following manufactures protocol (Invitrogen). The vectors were termed pENTR1-GG-gRNA1, pENTR1-GG-gRNA2, pENTR1-GG-gRNA3, etc. denoting their location in a finished Golden Gate assembled gRNA array.

Ten acceptor vectors were designed based on optimal type IIS restriction site overhangs as previously described (Cermak et al., 2011, Nucleic Acids Res 39(12):e82). These vectors contained a BsaI flanked region containing the LacZ coding region such that when a gRNA is inserted, the LacZ coding region is lost and thus blue/white colony selection can be implemented. These also contain a standard U6 promoter to drive the gRNA array and a terminating Csy4 sequence on the 3′ end and were ordered as gBLOCK fragments from IDT and included attB sequences on both ends for subsequent BP Clonase reaction using Gateway cloning, following manufactures protocol (Invitrogen Corp., Carlsbad, Calif.). The pENTR221 vector, modified to contain the spectinomycin coding region in place of the original kanamycin coding region, was used for cloning. These vectors were termed pENTR1-ACPT-GG1, pENTR1-ACPT-GG2, pENTR1-ACPT-GG3, etc., denoting the number of gRNAs to be incorporated via Golden Gate assembly.

### Design and Construction of Guide RNAs

Guide RNAs (gRNAs) were designed to the desired region of a gene using the CRISPR Design Program (Zhang Lab, MIT 2015; crispr.mit.edu). Multiple gRNAs were chosen based on the highest ranked values determined by off-target locations. The gRNAs were ordered in oligonucleotide pairs: 5′-overhang-G-gRNA sequence-3′ and 5′-AAAC-reverse complement gRNA sequence-C-3′. The gRNAs were cloned together using a modified version of the target sequence cloning protocol (Zhang Lab, MIT 2015; crispr.mit.edu). The oligonucleotide pairs were phosphorylated and annealed together using T4 PNK (New England Biolabs, Inc., Ipswich, Mass.) and 10×T4 Ligation Buffer (New England Biolabs, Inc., Ipswich, Mass.) in a thermocycler with the following protocol: 37° C. 30 minutes, 95° C. five minutes and then ramped down to 25° C. at 5° C./minute. pENTR1 vector backbones were digested with FastDigest BbsI (Fermentas, Thermo Fisher Scientific, Inc., Waltham, Mass.), FastAP (Fermentas, Thermo Fisher Scientific, Inc., Waltham, Mass.), and 10× Fast Digest Buffer and used for the ligation reaction. The digested pENTR1 vector was ligated together with the phosphorylated and annealed oligo duplex (dilution 1:200) from the previous step using T4 DNA Ligase and Buffer (New England Biolabs, Inc., Ipswich, Mass.). The ligation was incubated at room temperature for at least one hour and then transformed and mini-prepped (GeneJET Plasmid Miniprep Kit, Life Technologies). The plasmids were sequenced to confirm the proper insertion.

### Validation of Guide RNAs

Immortalized HSC1L cells were electroporated using the Neon Transfection System (100 μL Kit, Invitrogen Corp., Carlsbad, Calif.). Cells were counted and resuspended at a density of 1×10⁶ cells in 100 μL of R buffer. 2 μg of Cas9 plasmid, 2 μg of gRNA and 100 ng of GFP plasmid were added to the cell mixture. Cells were electroporated at 1400 V, 30 ms, one pulse. After transfection, cells were plated in a 2 mL culturing media in a 6-well plate. Cells were incubated for three days at 37° C. and then genomic DNA was collected using the GeneJET Genomic DNA Purification Kit (Thermo Fisher Scientific, Inc., Waltham, Mass.). Activity of the gRNAs was quantified by a Surveyor Digest, gel electrophoresis, and densitometry (Gushin et al., 2010, Meth Mol Biol 649:247-256).

Alternatively, HEK293T cells were maintained in DMEM medium supplemented with 10% fetal bovine serum (FBS). 1×10⁵ cells of HEK293T cells were seeded in 24-well plate the day before transfection. Transfection was performed using LIPOFECTAMINE 2000 (Invitrogen Corp., Carlsbad, Calif.), following the manufactures protocol. 500 ng of pT3.5-CAG-Csy4-T2A-hCas9, 250 ng of pENTR221-U6-gRNA, or 250 ng of pACPT array plasmid were diluted in 75 μL of OptiMEM (Thermo Fisher Scientific, Inc., Waltham, Mass.) and 5 μL of LIPOFECTAMINE 2000 was diluted in 75 μL of OptiMEM and then the mixtures were combined. The complete mixture was incubated for 15 minutes before being added to cells in a drop wise fashion. After 16 hours, the media was changed to fresh DMEM medium containing 10% fetal bovine serum. Cells were incubated for three days at 37° C. and then genomic DNA was collected using the GeneJET Genomic DNA Purification Kit (Thermo Fisher Scientific, Inc., Waltham, Mass.).

Activity of the gRNAs was quantified by a Surveyor nuclease digest, gel electrophoresis, and densitometry (Gushin et al., 2010, Meth Mol Biol 649:247-256).

For gene activation experiments, 250 ng of pT3.5-CAG-Csy4-T2A-dCas9-VP64, 250 ng of pT3.5-CAG-MS2-p65-HSF1-2A-eGFP, and 250 ng of pENTR221-U6-gRNA or pACPT array plasmid were transfected as above. Cells were incubated for three days at 37° C. and then RNA was extracted using PURELINK RNA Mini Kit (Thermo Fisher Scientific, Inc., Waltham, Mass.) and then reverse-transcribed by Transcriptor First Strand cDNA Synthesis Kit (Roche Molecular Systems, Inc., Pleasanton, Calif.).

Golden Gate Assembly of gRNA Arrays

Assembled single gRNAs were ligated into one vector via Golden Gate cloning (Engler et al., 2009. PLoS One, 4:e5553.). To make the arrays, 150 ng of each gRNA to be put into the array was combined with 150 ng of pACT vector, BsaI (New England Biolabs, Inc., Ipswich, Mass.), T4 DNA Ligase and Buffer (New England Biolabs, Inc., Ipswich, Mass.) and water. Each array was run in a thermocycler according to the following protocol: 37° C. for five minutes, 16° C. for ten minutes for ten cycles; 50° C. for five minutes; 80° C. for five minutes; and then cooled to 4° C. Each reaction was then combined with 1 μL of 25 mM ATP and 1 μL of Plasmid Safe and incubated for one hour at 37° C. The gRNA arrays were then transformed on kanamycin selection plates with X-gal and mini-prepped (GeneJET Plasmid Miniprep Kit, Life Technologies). The plasmids were sequenced to confirm the proper insertion.

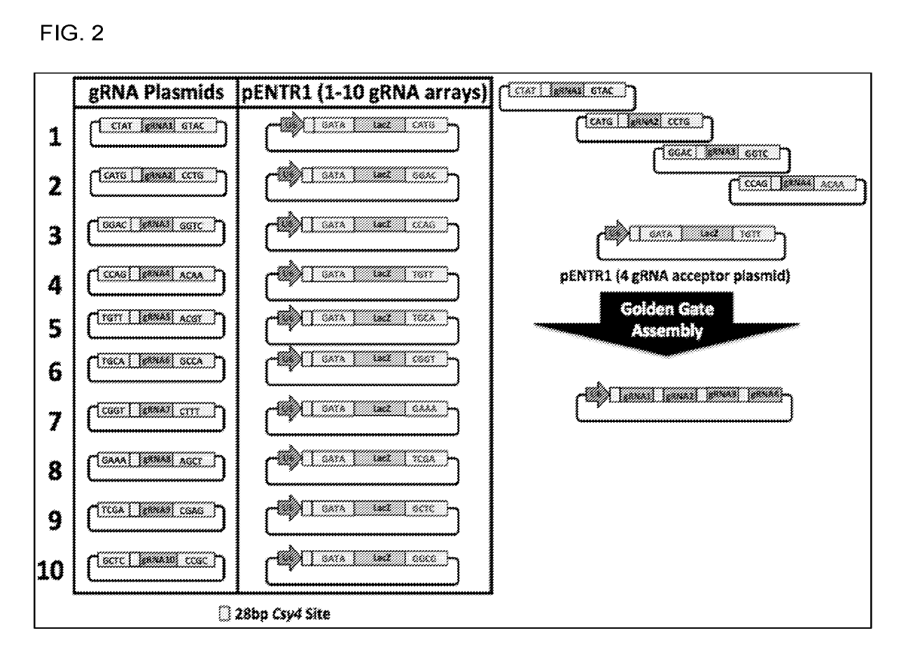

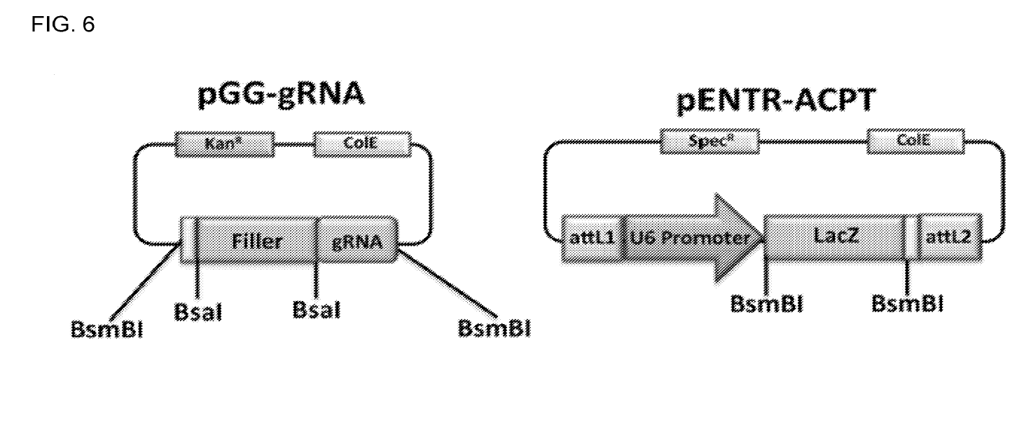

To generate gRNA arrays with multiple gRNAs, Golden Gate cloning was used with type IIS restriction enzyme sites (BsmBI) and overhangs previously published and validated for robust Golden Gate assembly of TALEN DNA binding domains (Cermak et al., 2011, Nucleic Acids Res 39(12):e82). Next, cassettes for oligonucleotide ligation of protospacer sequences using a different type IIS restriction enzyme (BsaI) flanking a stuffer sequence were designed and assembled. In addition, a Csy4 ribonuclease target sequence was included directly upstream of the target gRNA sequences such that after Golden Gate assembly each gRNA is directly flanked by the Csy4 target sequence (FIG. 6). Next, a gRNA array acceptor plasmid (pACPT) was designed containing a LacZ gene, for blue/white colony selection after Golden Gate assembly, flanked by appropriate BsmBI sites and an upstream U6 pol II promoter to drive expression of assembled gRNA arrays (FIG. 6). A terminal Csy4 target sequence—such that the last gRNA is free of additional sequence when processed—and a poly T termination sequence also were included. In order to produce a highly modular system for rapid and efficient cloning of the U6 driven gRNA array cassettes, attL1/2 sequences were included in pACPT for Gateway cloning (FIG. 1). The set of plasmids for oligonucleotide ligation are referred to as pGG 1-10 and the acceptor plasmids are referred to as pACPT 1-10 (FIG. 2). An example of the plasmids required for Golden Gate assembly of a four gRNA array and the structure of the final expression plasmid are shown in the right panel of FIG. 2.

Testing of Multiplex gRNA Arrays

293T cells were plated out at a density of 1×10⁵ cells per well in a 24-well plate. 150 μL of Opti-MEM medium was combined with 1.5 μg of single gRNA plasmid, 1.5 μg of Cas9 plasmid and 100 ng of GFP plasmid or 1.5 μg gRNA array, 1.5 μg Cas9-Csy4 and 100 ng of GFP plasmid. Another 150 μL of Opti-MEM medium was combined with 5 μl of LIPOFECTAMINE 2000 transfection reagent (Invitrogen Corp., Carlsbad, Calif.). The solutions were combined together and incubated for 10-15 minutes at room temperature. The DNA-lipid complex was added dropwise to one well of the 24-well plate. Cells were incubated for three days at 37° C. and then genomic DNA was collected using the GeneJET Genomic DNA Purification Kit (Thermo Fisher Scientific, Inc., Ipswich, Mass.). Activity of the single gRNA and the gRNA arrays was quantified by a Surveyor Digest, gel electrophoresis, and densitometry.

### Generation of Stable Cell Lines

One hundred thousand HEK293T cells were seeded into 24-well plates and allowed to adhere for eight hours. Cells were then transfected with 500 ng transposon plasmid and 500 ng PiggyBac7 hyperactive transposase expressing plasmid using LIPOFECTAMINE 2000 (Invitrogen Corp., Carlsbad, Calif.), following manufacturer's instructions. Two days post transfection cells were transferred to 10 cm plates and selected with 1 μg/mL puromycin for seven days to generate stable integration cell lines.

### Surveyor Nuclease Assay

Surveyor assays were performed as previously descried (Thermo Fisher Scientific, Inc., Ipswich, Mass.). Briefly, after electroporation of CRISPR/Cas9 plasmids and incubation for three days genomic DNA was extracted using GeneJET Genomic DNA Purification Kit (Thermo Fisher Scientific, Inc., Ipswich, Mass.), following the manufacturer's instructions. PCR amplicons were generated spanning the Cas9 binding site using ACCUPRIME Taq HF (Invitrogen Corp., Carlsbad, Calif.) using the following PCR cycle: initial denaturation at 95° C. for five minutes; 40× (95° C. for 30 seconds, 55° C. or 60° C. for 30 seconds, 68° C. for 40 seconds); final extension at 68° C. for two minutes. PCR amplicons were denatured and annealed as follows: 95° C. for five minutes, 95-85° C. at −2° C./s, 85-25° C. at −0.1° C./s, 4° C. hold. Primer sequences can be found in Table 1, below.

TABLE 1
Primers

| gRNA target | | SEQ ID |
|---|---|---|
| sequence primers | | NO |
| GOSR1 | GACAGAATGTTTGAGACAA | 3 |
| PPP2R2A | GAGGTAGGCAGATTACCAA | 4 |
| CNTFR | GCGTAGACAACTGCGGCGG | 5 |
| DMD | TTATGGCCTAGCTGAGAAG | 6 |
| ZBTB10 | ATGTCAGCATTGTGGTAAG | 7 |
| KAT7 | GCTTAGCCTGGCTGAGGAG | 8 |
| SPPL3 | GCTGGAGACGTCAAAGTGC | 9 |
| CCM2 | GTCAGTTAACGTCCATACC | 10 |
| PRDX1 | CCACAGCTGTTATGCCAGA | 11 |
| TRIP12 | GTCACTGCGACGTTCACAG | 12 |
| HBG1 | GGCTAGGGATGAAGAATAAA | 13 |
| IL1B | AAAAACAGCGAGGGAGAAAC | 14 |
| ASCL1 | GCAGCCGCTCGCTGCAGCAG | 15 |
| MYOD1 | GGGCCCCTGCGGCCACCCCG | 16 |
| IL-1R2 | GACCCAGCACTGCAGCCTGG | 17 |
| POU5F1(OCT4) | GGGGGGAGAAACTGAGGCGA | 18 |
| KLF4 | ATGGGAGAAGGCGGAGGAAA | 19 |
| NALCN | GGGACTGCAGTGATGCCGAA | 20 |
| LIN28A | GGGGCTGCCCGCGGGGGGTT | 21 |
| ZFP42(REX1) | GGGTCTTGGGAGGGGGCGCA | 22 |
| Cel1 primers | | |
| GOSR1 Forward | GATCGTTTCTCACAGACCCTATA | 23 |
| GOSR1 Reverse | ATTCAAGTGGTGTGGGGAGG | 24 |
| PPP2R2A Forward | TCGGCTATGTGACATGAGGG | 25 |
| PPP2R2A Reverse | CAAGGTTACAGAGCCCAACC | 26 |
| CNTFR Forward | CCCGGTTTCTCCCAACAGAT | 27 |
| CNTFR Reverse | GTCAGCATTCGACCACCCTA | 28 |
| DMD Forward | CCTTCTCACTGTCTTCGGGT | 29 |
| DMD Reverse | AATGCCTGATCACGTGCATC | 30 |
| ZBTB10 Forward | AGAGGAGGGGTACTGTGACT | 31 |
| ZBTB10 Reverse | ATAGCTGGCCACGGTCATAA | 32 |
| KAT7 Forward | ACAGAGGAATGCAGGCAGTA | 33 |
| KAT7 Reverse | ATCCGCAGTTCCTTTGGGT | 34 |
| SPPL3 Forward | TCCCCAACTCCTCCTTGAAC | 35 |
| SPPL3 Reverse | AGATAAACAGCAGGCCAGGG | 36 |
| CCM2 Forward | CCTGGTGGCCTGAGTATGAA | 37 |
| CCM2 Reverse | AATGTGATGGGACTGGCTCA | 38 |
| PRDX1 Forward | AGGTGAAGGCTGCTGGTTAT | 39 |
| PRDX1 Reverse | AGAAGTGGTTTGGTCCTAGGA | 40 |
| TRIP12 Forward | AGCATGGGTGAAGGCTGTAA | 41 |
| TRIP12 Reverse | ACCTGGCTCACAAATCAGGA | 42 |

Three microliters of the annealed amplicon was then diluted with 6 μL of 1× ACCUPRIME PCR buffer and treated with 1 μL of Surveyor nuclease with 1 μL of enhancer (Thermo Fisher Scientific, Inc., Ipswich, Mass.) at 42° C. for 20 minutes. The reaction was then stopped by adding 3 μL of 15% Ficol-400 and 0.05% Orange G solution containing 1 mM EDTA and subsequently run on a standard 10% TBE gel. Percent gene modification was calculated using Image J software as described (Thermo Fisher Scientific, Inc., Ipswich, Mass.).

### Q-RT-PCR Analysis

Taq-man quantitative PCR was performed with following primer and probes. ASCL1; Hs00269932_m1, MYOD1; Hs02330075_g1, HBG1/HBG2; Hs00361131_g1, IL1B; Hs01555410_m1, IL1R2; Hs01030384_m1, ACTB Hs99999903_m1. (Thermo Fisher Scientific, Inc. Ipswich, Mass.).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A polynucleotide for modulating transcription from a plurality of genomic targets, the polynucleotide comprising:
a polynucleotide encoding a gRNA array comprising:
a polynucleotide encoding a first gRNA targeted to a first genomic target; and
a polynucleotide encoding a second gRNA targeted to a second genomic target;
the polynucleotide encoding the first gRNA and the polynucleotide encoding the second gRNA operably linked to an inducible regulatory sequence; and
a polynucleotide sequence encoding a nuclease-deficient Cas9 polypeptide.

2. The polynucleotide of claim 1 further comprising an enzyme cleavable linker sequence linking the polynucleotide encoding the first gRNA and the polynucleotide encoding the second gRNA.

3. The polynucleotide of claim 1 wherein the nuclease-deficient Cas9 polypeptide comprises a fusion polypeptide comprising a transcription activating domain.

4. The polynucleotide of claim 3 wherein the transcription activating domain comprises VP64.

5. The polynucleotide of claim 1 wherein the nuclease-deficient Cas9 polypeptide comprises a transcription repressing domain.

6. The polynucleotide of claim 5 wherein the transcription repressing domain comprises a Krüppel associated box domain.

7. The polynucleotide of claim 1 wherein the gRNA array comprises at least 5 gRNAs.

8. A method of modulating expression of a plurality of genomic target coding regions in a cell, the method comprising:
introducing into the cell the polynucleotide of claim 1, wherein gRNAs in the array target the genomic target coding regions; and
inducing transcription of the gRNA array.

9. The method of claim 8 wherein expression of two or more genomic target coding regions are modulated simultaneously.

10. The method of claim 8 further comprising screening the modulated expression of the genomic target coding regions for a change in phenotype.

11. The method of claim 8 further comprising identifying mRNA targets of a particular phenotype.

12. The method of claim 8 further comprising identifying causal cancer genes.

13. The method of claim 8 further comprising overexpressing a genomic target coding region that encodes a polypeptide of interest.

14. The method of claim 13 further comprising isolating at least a portion of the polypeptide of interest.

15. The method of claim further comprising altering biochemical pathways to favor biosynthesis of a compound of interest.

16. The method of claim 15 further comprising isolating at least a portion of the compound of interest.

17. The method of claim 8 further comprising generating a synthetic CRISPR immune system to increase resistance of the cell to infection by a virus.

18. The method of claim further comprising activating a cellular pathway in a therapeutic cell to increase the therapeutic cell's therapeutic activity.

19. A method for generating a genetically modified organism, the method comprising:
introducing into cells of the organism the polynucleotide of claim 1, wherein gRNAs in the array target the genomic target coding regions; and
inducing transcription of the gRNA array.

20. The method of claim 19 wherein the organism is a mouse.


SEQUENCE LISTING (42 sequences)

SEQ ID NO: 1 - RNA, 20 nt, artificial
  Cys4 binding site
guucacugccguauaggcag

SEQ ID NO: 2 - RNA, 28 nt, artificial
  Cys4 binding site
guucacugccguauaggcagcuaagaaa

SEQ ID NO: 3 - DNA, 19 bp, artificial
  Primer
gacagaatgtttgagacaa

SEQ ID NO: 4 - DNA, 19 bp, artificial
  Primer
gaggtaggcagattaccaa

SEQ ID NO: 5 - DNA, 19 bp, artificial
  Primer
gcgtagacaactgcggcgg

SEQ ID NO: 6 - DNA, 19 bp, artificial
  Primer
ttatggcctagctgagaag

SEQ ID NO: 7 - DNA, 19 bp, artificial
  Primer
atgtcagcattgtggtaag

SEQ ID NO: 8 - DNA, 19 bp, artificial
  Primer
gcttagcctggctgaggag

SEQ ID NO: 9 - DNA, 19 bp, artificial
  Primer
gctggagacgtcaaagtgc

SEQ ID NO: 10 - DNA, 19 bp, artificial
  Primer
gtcagttaacgtccatacc

SEQ ID NO: 11 - DNA, 19 bp, artificial
  Primer
ccacagctgttatgccaga

SEQ ID NO: 12 - DNA, 19 bp, artificial
  Primer
gtcactgcgacgttcacag

SEQ ID NO: 13 - DNA, 20 bp, artificial
  Primer
ggctagggatgaagaataaa

SEQ ID NO: 14 - DNA, 20 bp, artificial
  Primer
aaaaacagcgagggagaaac

SEQ ID NO: 15 - DNA, 20 bp, artificial
  Primer
gcagccgctcgctgcagcag

SEQ ID NO: 16 - DNA, 20 bp, artificial
  Primer
gggcccctgcggccaccccg

SEQ ID NO: 17 - DNA, 20 bp, artificial
  Primer
gacccagcactgcagcctgg

SEQ ID NO: 18 - DNA, 20 bp, artificial
  Primer
ggggggagaaactgaggcga

SEQ ID NO: 19 - DNA, 20 bp, artificial
  Primer
atgggagaaggcggaggaaa

SEQ ID NO: 20 - DNA, 20 bp, artificial
  Primer
gggactgcagtgatgccgaa

SEQ ID NO: 21 - DNA, 20 bp, artificial
  Primer
ggggctgcccgcggggggtt

SEQ ID NO: 22 - DNA, 20 bp, artificial
  Primer
gggtcttgggagggggcgca

SEQ ID NO: 23 - DNA, 23 bp, artificial
  Primer
gatcgtttctcacagaccctata

SEQ ID NO: 24 - DNA, 20 bp, artificial
  Primer
attcaagtggtgtggggagg

SEQ ID NO: 25 - DNA, 20 bp, artificial
  Primer
tcggctatgtgacatgaggg

SEQ ID NO: 26 - DNA, 20 bp, artificial
  Primer
caaggttacagagcccaacc

SEQ ID NO: 27 - DNA, 20 bp, artificial
  Primer
cccggtttctcccaacagat

SEQ ID NO: 28 - DNA, 20 bp, artificial
  Primer
gtcagcattcgaccacccta

SEQ ID NO: 29 - DNA, 20 bp, artificial
  Primer
ccttctcactgtcttcgggt

SEQ ID NO: 30 - DNA, 20 bp, artificial
  Primer
aatgcctgatcacgtgcatc

SEQ ID NO: 31 - DNA, 20 bp, artificial
  Primer
agaggaggggtactgtgact

SEQ ID NO: 32 - DNA, 20 bp, artificial
  Primer
atagctggccacggtcataa

SEQ ID NO: 33 - DNA, 20 bp, artificial
  Primer
acagaggaatgcaggcagta

SEQ ID NO: 34 - DNA, 19 bp, artificial
  Primer
atccgcagttcctttgggt

SEQ ID NO: 35 - DNA, 20 bp, artificial
  Primer
tccccaactcctccttgaac

SEQ ID NO: 36 - DNA, 20 bp, artificial
  Primer
agataaacagcaggccaggg

SEQ ID NO: 37 - DNA, 20 bp, artificial
  Primer
cctggtggcctgagtatgaa

SEQ ID NO: 38 - DNA, 20 bp, artificial
  Primer
aatgtgatgggactggctca

SEQ ID NO: 39 - DNA, 20 bp, artificial
  Primer
aggtgaaggctgctggttat

SEQ ID NO: 40 - DNA, 21 bp, artificial
  Primer
agaagtggtttggtcctagga

SEQ ID NO: 41 - DNA, 20 bp, artificial
  Primer
agcatgggtgaaggctgtaa

SEQ ID NO: 42 - DNA, 20 bp, artificial
  Primer
acctggctcacaaatcagga